(19) United States
(12) Patent Application Publication
(10) Pub. No.: US 2024/0058486 A1
(43) Pub. Date: Feb. 22, 2024

(54) COMPACT MOBILE UVC LIGHT PROJECTION UNIT

(71) Applicant: UVC Science, Inc., Reno, NV (US)

(72) Inventor: Jozef Richelieu, Reno, NV (US)

(21) Appl. No.: 18/232,126

(22) Filed: Aug. 9, 2023

Related U.S. Application Data
(60) Provisional application No. 63/398,057, filed on Aug. 15, 2022.
(60) Provisional application No. 63/483,295, filed on Feb. 5, 2023.

(51) Int. Cl. A61L 2/10

(52) U.S. Cl. CPC A61L 2/10; A61L 2202/11; A61L 2202/16

(57) ABSTRACT

A compact UVC light projection unit for providing UVC illumination comprises a housing, a plurality of UVC light sources supported by the housing, and a handle connected to the housing for holding the housing. The plurality of UVC light sources comprises UVC light emitting diodes (LEDs) configured to emit light having a wavelength in the range of 260 to 280 nm. The UVC light projection unit may further comprise a fan and/or a plurality of visible light sources.

### RELATED APPLICATIONS

This application claims priority benefit of U.S. Provisional Application No. 63/398,057, filed Aug. 15, 2022, titled “TACTICAL HANDHELD UV LED PROJECTION UNIT” and U.S. Provisional Application No. 63/483,295, filed Feb. 5, 2023, titled “COMPACT MOBILE UVC LIGHT PROJECTION UNIT”. The entire contents of each of the applications listed in this paragraph are hereby incorporated herein by reference.

### TECHNICAL FIELD

The present application generally relates to apparatus for projecting ultraviolet light and in particular ultraviolet light projection units providing UVC illumination, for example, for sterilization and/or inactivation of various viruses and bacteria.

### DESCRIPTION OF THE RELATED TECHNOLOGY

The need for sterilization and inactivation of viruses was apparent during the recent COVID pandemic. However, applications involving sterilization and inactivation extend beyond this context. More generally mobile tools that provide the ability to combat infectious viruses and disease carrying bacteria such as in hospitals, airplanes, and other public areas is certainly desirable. Other scenarios, such as biohazard cleanup, sanitizing military equipment contaminated due to germ warfare, etc., may also benefit from effective tools for destroying or at least degrading the potency of certain biological contaminants.

### SUMMARY OF THE INVENTION

Various designs described herein may potentially provide for reducing the activity of viruses and/or bacteria on surfaces. One example design comprises a UVC light projection unit for providing UVC illumination. The UVC light projection unit comprises a housing having a front and back, top and bottom and sides and a plurality of light sources supported by the housing. The plurality of light sources comprises a first group of light sources comprising a plurality of UVC light emitting diodes (LEDs) and a second group of light sources comprising a plurality of visible light emitting diodes configured to project UVC light and visible light forward. The UVC light projection unit further comprises a handle closer to the top of the housing than the bottom or sides, the handle for holding the housing such that the plurality of light sources project light in a forward direction. The UVC light projection unit additionally comprise a fan disposed to provide cooling for said plurality of light sources.

### BRIEF DESCRIPTION OF THE DRAWINGS

FIG. 1 perspective view of an example UVC light projection unit comprising a plurality of light sources. The plurality of light sources include UVC light sources comprising UVC light emitting diodes (LEDs). A plurality of visible LEDs are also included.

FIG. 2 is another perspective view of the example UVC light projection unit of FIG. 1 showing a back view. A UVC light projection unit includes a fan for providing cooling to the plurality of LEDs.

FIG. 3 is a plot on axis of intensity (in relative units) and wavelength (in nanometers) showing the wavelength distribution of light output by the UVC LED.

FIG. 4 is cross-sectional view of a UVC LED and an optical element comprising a lens as well as a reflective surface, the optical element being disposed to receive light from the UVC LED. The optical element is configured to reduce divergence of UVC light from said UVC LED.

FIG. 5 is cross-sectional view of a visible LED and an optical element similar to the optical element of FIG. 4 disposed to receive light from the visible LED. The optical element is configured to reduce divergence of visible light from said visible LED.

FIG. 6 is perspective view of the optical element of FIGS. 4 and 5. In this example, the optical element has an outer surface having a hexagonal pattern thereon.

FIG. 7 is another perspective view of the optical element of FIGS. 4 and 5 showing a concave (e.g., parabolic) surface reflective to UVC light.

### DETAILED DESCRIPTION

Ultraviolet (UV) light includes wavelengths from 100 nm to 400 nm. This range is often partitioned into subranges such as UV-A, which may be considered to span 315 nm to 400 nm, UV-B, which may be considered to be from 280 nm to 315 nm in wavelength, and UV-C (or UVC as used herein), which may be considered to be from 200 nm to 280 nm.

As discussed herein, UVC light can be used to sterilize surfaces as UVC light kills or degrades the potency of various harmful microorganisms that may be on those surfaces. UVC light attacks nucleic acids and damages the DNA of the microorganism. Accordingly, technology described herein, may be used to partially or fully sterilize and/or render inactive viruses and/or bacteria on surfaces from a distance of a 1 to 3 feet, possibly larger.

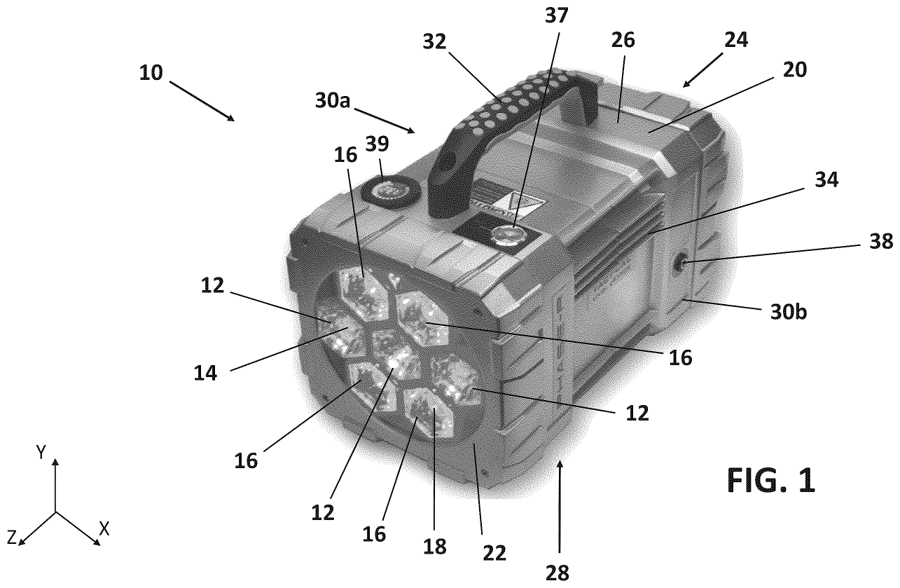

FIG. 1 shows an example UVC light projection unit 10 comprising a plurality of light sources 12, 16. In the example shown, the plurality of light sources 12, 16 comprises a first group of light sources 12 comprising a plurality of UVC light emitting diodes (LEDs) 14 and a second group of light sources 16 comprising a plurality of visible light emitting diodes 18. In the example shown, the middle row comprises the plurality of UVC light sources 12 and the top and bottom rows or in this case pairs of light sources are the visible light sources. In this example, therefore, the plurality of light sources comprise three UVC light sources 12 and four visible light sources. Other designs, however, may have different distribution of UVC and visible light source in number, location, or both.

The plurality of UVC light sources 12 and UVC LEDs 14 are configured to emit UVC light capable of destroying, disabling or weakening virus and bacteria. This UVC light may, for example, comprise light within the range of from 260 nm to 280 nm, for example, 265 nm in wavelength. In various implementations, most, all or nearly all of the light emitted from the UVC LEDs 14 and the corresponding UVC light source 12 may comprise light in the wavelength range of 250 nm to 290 nm, for example, from 250, 252, 253, 254, 255, 256, 257, 258, 259, 260, 261, 262, 263, 264, or 265 to 266, 267, 268, 269, 270, 271, 272, 273, 274, 275, 277, 280, 282, 285 nm, 286, 287 nm or any range formed by any of these values such as 253 to 287 nm, 260 to 270 nm or 263 nm to 267 nm, or 265 nm to 275 nm or at 265 nm. The plurality of UVC light sources 12 and/or UVC LEDs 14 may, for example, be configured to emit more than 50%, 60%, 70%, 80%, 90%, 95%, 97%, 98% or 99% of the light output therefrom in any range formed by any of these percentages, in the wavelength range from 250, 252, 253, 254, 255, 256, 257, 258, 259, 260, 261, 262, 263, 264 or 265 to 266, 267, 268, 269, 270, 271, 272, 273, 274, 275, 277, 280, 282, 285 nm, 287 nm or any range between any of these wavelengths such as from 250 nm to 280 nm, or 253 to 287 nm, or 255 to 285 nm, or 260 nm to 280 nm or 260 to 270 nm or 263 nm to 267 nm, 265 nm to 275 nm or at 265 nm. Similar, the plurality of UVC light sources 12 and/or UVC LEDs 14 may, for example, have a spectral peak, e.g., a central spectral peak, for light output therefrom in the wavelength range from 250, 252, 253, 254, 255, 256, 257, 258, 259, 260, 261, 262, 263, 264 or 265 to 266, 267, 268, 269, 270, 271, 272, 273, 274, 275, 277, 280, 282, 285 nm, 287 nm or any range between any of these values such as from 250 nm to 280 nm, or 253 to 287 nm, or 255 to 285 nm, or 260 nm to 280 nm or 260 to 270 nm or 263 nm to 267 nm, 265 nm to 275 nm or at 265 nm.

The second group of light sources 16 comprising a plurality of visible light emitting diodes 18 may comprise color LEDs that emit color light. Such color light may make the user aware that UV light is being radiated from the UVC light sources 12/UVC LEDs 14, which may provide for safer operation and may possibly inform the user of where UVC light may be being projected by the UVC light projection unit 10. In some example designs, the visible light LEDs 16 comprises blue LEDs. Other color LEDs may be used. For example, red LEDs, purple LEDs, green LEDs, orange LEDs, yellow LEDs or other color LEDs may be employed in different designs.

The plurality of light sources 12, 16 and corresponding LEDs 14, 18 are supported by a housing 20. This housing 20 may comprise metal such as aluminum (e.g., anodized aluminum) in some designs. For example, some implementations are design to satisfy military specifications. The housing may additionally or alternatively comprise plastic such as polycarbonate. This housing 20 may house electronics and/or electrical connections (e.g., wiring) configured to drive the UVC and/or visible LEDs 14, 18.

This UVC light projection unit 10 and housing 20 has a front 22 and a back 24. The light sources 12, 16 and corresponding LEDs 14, 18 are disposed on the front 22 of the housing 20. UVC projection unit 10 and housing 22 also has a top 26 and bottom 28 and sides 30a, 30b. In the illustrated design, the top 26 and bottom 28 have a length and width (parallel, respectively, to the Z and X directions in the XYZ coordinate system in the lower left), with the length longer than the width. Similarly, the sides 30a, 30b have a length and height (parallel to the Z and Y directions, respectively), with the length longer than the height. In some implementations, the front 22 and back 24 have a width (parallel to the X direction) corresponding to the width of the top 26 and bottom 28 and have a height (parallel to the Y direction) corresponding to the height of the sides 30a, 30b. Additionally, in various implementations, the area of the front 22 is smaller than the area of the top 26, smaller than the area of the one of the sides 30a, 30b, smaller than the area of bottom 28 or any combination of these. Additionally, in various implementations, the area of the back 24 is smaller than the area of the top 26, smaller than the area of the one of the sides 30a, 30b, smaller than the area of bottom 28 or any combination of these. Accordingly, in some designs, the housing 20 has the shape of a rectangular prism, possibly with beveled and/or smoothed corners. In some case, for example, the top 26, bottom 28 and sides 30a, 30b are rectangular, possibly with beveled and/or smoothed corners, or at least more rectangular, and the front 22 and back 24 are square or rectangular, possibly with beveled and/or smoothed corners, or at least more square.

In various implementations, the length of the top 26 and bottom 28 as well as sides 30a, 30b extends in a longitudinal direction (parallel to the Z direction) corresponding to the forward direction. The light output from the plurality of light sources 12, 16 together projects mostly in this forward direction.

As illustrated, the plurality of light sources (e.g., the UVC light sources and the visible light sources) 12, 16 are at the front 22 of the UVC light projection unit. Likewise, the plurality of light sources (e.g., the UVC light sources and the visible light sources) 12, 16 are configured to point forward, for example, along the longitudinal direction (e.g., parallel to the Z axis).

In various implementations, the plurality of light sources, for example, the UVC light sources and the visible light sources together, 12, 16 are arranged in an array. In the example shown in FIG. 1, plurality of the UVC light sources 12 and the UVC LEDs 14 together are arranged in a hexagonal array. Other types of arrays or arrangements, however, can be used in the design.

As illustrated, the individual UV light sources 12, 14 shown in FIG. 1 have a hexagonal cross-sections or apertures (e.g., output aperture). Accordingly, in various implementations, either or both the UV light sources 12 and/or the visible light sources 16 have hexagonal cross-sections or apertures (e.g., output apertures). Other shapes are possible.

In some implementations, the array may comprise at least 3 rows of light sources 12, 16 and/or LEDs 14, 18 and may comprise, for example, 1 row, 2 rows, 3 rows, 4 rows, 5 rows, or any range formed by any of these values and possibly more rows. As illustrated, the rows are parallel to the X direction in this example. In various implementations, the array may comprise at least 3 columns of light sources 12, 16 and/or LEDs 14, 18 and may comprise, for example, 2 columns, 3 columns, 4 columns, 5 columns, or any range formed by any of these values and possibly more columns. As illustrated, the columns are parallel to the Y direction in this example.

In various implementations, the UVC light projection unit 10 comprises at least 1, 2, 3, 4, 5, 6, 7, 8, 9, or 10 UVC light sources 12 and/or UVC LEDs 14 or any number in any range formed by any of these values or possibly more. In some designs, most, if not all, of these UVC light sources 12 and/or UVC LEDs 14 are directed forward and project most light, or possibly at least 20%, 30%, 40%, 50%, 55%, 60%, 65%, 70%, 75%, 80%, 85%, 90%, 95%, or 100% of the light or any range formed by any of these values, forward (e.g., mostly in the Z direction as shown by the XYZ coordinate system depicted in the lower left-hand corner of FIG. 1) or within an angular range of ±10°, ±15°, ±20°, ±22.5°, ±30°, ±40°, ±45°, ±50°, ±60°, ±70°, or ±75° of the forward (Z) direction or any range formed by any of these values or possibly larger or smaller such as from ±20° to ±50° of the forward (Z) direction. In various designs, the UVC light sources 12 include a lens and/or a reflector 22 configured to direct most of the light, or possibly at least 20%, 30%, 40%, 50%, 55%, 60%, 65%, 70%, 75%, 80%, 85%, 90%, 95%, 100% of the light or any range formed by any of these values, emitted from the respective UVC LED 14 forward (e.g., mostly in the Z direction) or within the angular range thereof (e.g., within ±10°, ±15°, ±20°, ±22.5°, ±30°, ±40°, ±45°, ±50°, ±60°, ±70°, or ±75° of the forward direction, e.g., Z direction or any range formed by any of these values or possibly larger or smaller, such as from ±20° to ±50°). Likewise, in various implementations, the individual UVC light sources 12 include respective lenses and/or reflectors 22 having respective optical axes directed in the forward (e.g., Z) direction.

The lens may comprise, for example, a fused silica lens. The lens may have positive optical power. In some cases, the lens is a plane convex lens. The lens may be configured to collimate light from the UVLED. For example, the lens may be positioned at a distance from the UVLED equal to the focal length of the lens. In other implementations, this distance may be larger or smaller that the focal length. In some implementations, the lens comprises an aspheric lens having an aspheric refractive surface. In various implementations is optically transmissive to 220-290 nm wavelengths. The lens may have a short focal length, e.g., 4, 4.5, 5, 5.5, 6, 6.5, 7, 7.5, 8, 8.5, 9, 9.5, 10 mm, 11 mm, or 12 mm or any range between any of these values or possibly higher or lower. In certain designs, the lens is 5-12, 6-11 mm or 7-10 mm or 8-9 mm from the visible LED 18 or any range between any of these values or possibly farther or closer. In some designs, the lens is a focal length away from the visible LED 18 or thereabouts. The lens may be inserted into a module retained by an outer end cap.

As discussed above, in various designs, one or more UVC light sources 12 and possibly one or more visible light sources 16 comprise a lens that receives light from the UVC LED and/or visible LED 14, 18. The lens may reduce the divergence of and/or potentially collimate light emitted by the UVC LED and/or visible LED 14, 18. The lens comprises optically transmissive material that is optically transmissive to UVC light output by the UVC LED 14 such as in the range of 250 nm to 290 nm, 250 nm to 280 nm or 260 nm to 290 nm, or 260 nm to 280 nm or any range between any of these values or possibly larger or smaller.

In some implementations, the lens comprises fused silica. In various implementations, the lens may comprise fused silica glass having a transmittance (e.g., internal transmittance or transmittance corrected to eliminate the effects of scattering and of reflection from surfaces) of UVC light with 245-280 nm wavelength of least 95% for a 10 mm thickness of the fused silica glass although in other implementations this transmittance is at least 50%, 55%, 60%, 65%, 70%, 75%, 80%, 85%, 90%, 94%, 96%, 98%, 99% or 100% or any range formed by any of these values or possibly more or less. In various implementations, the OH (e.g., Hydroxyl) content is not larger than 5 ppm although in other implementations the OH content is not larger than 0.05 ppm, 0.1 ppm, 0.5 ppm, 1 ppm, 2 ppm, 3 ppm, 4 ppm, 6 ppm, 8 ppm, 10 ppm, 20 ppm, 30 ppm, 40 ppm, 50 ppm, 60 ppm, 70 ppm, 80 ppm, 90 ppm, 100 ppm, 125 ppm, 150 ppm, 200 ppm, 300 ppm, 400 ppm, 500 ppm or any range formed by any of these values or possibly more or less. Additionally, in various implementations, a content of Li, Na, K, Mg, Ca and Cu each are smaller than 0.1 ppm although in some implementations the content of any one or more possibly each of Li, Na, K, Mg, Ca and Cu are smaller than 0.001 ppm, 0.005 ppm, 0.01 ppm, 0.05 ppm, 0.2 ppm, 0.3 ppm, 0.4 ppm, 0.5 ppm, 0.6 ppm, 0.7 ppm, 0.8 ppm, 0.9 ppm, 1 ppm, 1.25 ppm, 1.50 ppm, 2.00 ppm, 3.00 ppm, 4.00 ppm, or 5.00 ppm or any range formed by any of these values or possibly more or less.

In some implementations, the glass has a viscosity coefficient at 1215° C. of at least 10^{11.5} Pa·s; and a Cu ion diffusion coefficient of not larger than 1×10⁻¹⁰ cm²/sec in a depth range of greater than 20 μm up to 100 μm, from the surface, when leaving to stand at 1050° C. in air for 24 hours. However, the glass need not be so limited as other implementations are possible.

In some case, the glass may be fabricated by crystobalitizing powdery silica raw material and then, fusing the crystobalitized silica material in a non-reducing atmosphere. However, the method of manufacture should not be so limited.

In some implementations, the fused silica glass may exhibit a high transmittance of ultraviolet, visible and infrared rays, may have high purity and heat resistance, and may exhibits a reduced diffusion rate of metal impurities or any combination of these traits.

In various implementations, the UVC light projection unit 10 comprises at least 1, 2, 3, 4, 5, 6, 7, 8, 9, or 10 visible light sources 16 and/or visible LEDs 12 or any number in any range formed by any of these values or possibly more. In some designs, most, if not all, of these visible light sources 16 and/or visible LEDs 18 are directed forward and project most light, or possibly at least 20%, 30%, 40%, 50%, 55%, 60%, 65%, 70%, 75%, 80%, 85%, 90%, 95%, 100% of the light or any range formed by any of these values, forward or within an angular range of ±10°, ±15°, ±20°, ±22.5°, ±30°, ±40°, ±45°, ±50°, ±60°, ±70°, or ±75° of the forward (Z) direction or any range formed by any of these values or possibly larger or smaller such as from ±20° to ±50° of the forward (Z) direction. In various designs, the visible light sources 16 include a lens and/or a reflector configured to direct most or possibly at least 20%, 30%, 40%, 50%, 55%, 60%, 65%, 70%, 75%, 80%, 85%, 90%, 95%, 100% of the light emitted from the respective visible LED 18 of the light or any range formed by any of these values, forward (e.g., mostly in the Z direction) or with the angular range thereof (e.g., within ±10°, ±15°, ±20°, ±22.5°, ±30°, ±40°, ±45°, ±50°, ±60°, ±70°, or ±75° of the forward direction or Z direction or any range formed by any of these values or possibly larger or smaller, such as from ±20° to ±50°). Likewise, in various implementations, the individual visible light sources 16 include respective lenses and/or reflectors having respective optical axes directed in the forward direction (e.g., Z direction).

The lens may comprise, for example, a fused silica lens. Accordingly, in some implementations, the lens comprises fused silica. In various implementations, the lens may comprise fused silica glass having a transmittance (e.g., internal transmittance or transmittance corrected to eliminate the effects of scattering and of reflection from surfaces) of UVC light with 245-280 nm wavelength of least 95% for a 10 mm thickness of the fused silica glass although in other implementations this transmittance is at least 50%, 55%, 60%, 65%, 70%, 75%, 80%, 85%, 90%, 94%, 96%, 98%, 99%, 99.9%, or 100% or any range formed by any of these values or possibly more or less. In various implementations, the OH (e.g., Hydroxyl) content is not larger than 5 ppm although in other implementations the OH content is not larger than 0.05 ppm, 0.1 ppm, 0.50 ppm, 1 ppm, 2 ppm, 3 ppm, 4 ppm, 10 ppm, 20 ppm, 30 ppm, 40 ppm, 50 ppm, 60 ppm, 70 ppm, 80 ppm, 90 ppm, 100 ppm, 125 ppm, 150 ppm, 200 ppm, 300 ppm, 400 ppm, 500 ppm or any range formed by any of these values or possibly more or less. Additionally, in various implementations, a content of Li, Na, K, Mg, Ca and Cu each are smaller than 0.1 ppm although in some implementations the content of any one or more possibly each of Li, Na, K, Mg, Ca and Cu are smaller than 0.001 ppm, 0.005 ppm, 0.01 ppm, 0.05 ppm, 0.2 ppm, 0.3 ppm, 0.4 ppm, 0.5 ppm, 0.6 ppm, 0.7 ppm, 0.8 ppm, 0.9 ppm, 1 ppm, 1.25 ppm, 1.50 ppm, 2.00 ppm, 3.00 ppm, 4.00 ppm, 5.00 ppm or any range formed by any of these values or possibly more or less.

In some case, the glass may be fabricated by crystobalitizing powdery silica raw material and then, fusing the crystobalitized silica material in a non-reducing atmosphere. However, the method of manufacture should not be so limited.

The lens may have positive optical power. In some cases, the lens is a plane convex lens. The lens may be configured to collimate light from the LED. For example, the lens may be positioned at a distance from the LED equal to the focal length of the lens. In other implementations, this distance may be larger or smaller that the focal length. In some implementations, the lens comprises an aspheric lens having an aspheric refractive surface. In various implementations is optically transmissive to 220-290 nm wavelengths. The lens may have a short focal length, e.g., 4, 4.5, 5, 5.5, 6, 6.5, 7, 7.5, 8, 8.5, 9, 9.5, 10 mm, 11 mm, 12 mm or any range between any of these values or possibly higher or lower. In certain designs, the lens is 5-12, 6-11 mm or 7-10 mm or 8-9 mm from the visible LED 18 or any range between any of these values or possibly farther or closer. In some designs, the lens is a focal length away from the visible LED 18 or thereabouts. The lens may be inserted into a module retained by an outer end cap.

In various implementations, therefore, the total number of light sources 12, 16 (UVC and visible) and correspondingly the total number of UVC and visible LEDs 14, 18 comprise at least 2, 3, 4, 5, 6, 7, 8, 9, 10, 12, 15, 18, or 20 light sources and/or LEDs or any number in any range formed by any of these values or possibly more. As discussed above, these light sources 12, 16 and corresponding LEDs 14, 18 may be directed forward (mostly along the Z direction) and/or may project light centered in that forward direction. These light sources 12, 16, and LEDs 14, 18 may be arranged in an array such as a hexagonal array, although other arrangements are possible.

The plurality of UVC light sources 12 and thus the mobile UVC light projection unit 10 may be capable of outputting radiant flux in the amount from 50 to 2500 mW or possibly higher or lower depending, e.g., on the design. Likewise, the UVC light sources 12 and thus the mobile UVC light projection unit 10 may be capable of outputting (e.g., radiant flux) in the amount of 10, 20, 30, 40, 50, 75, 100, 125, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2800, 3000, 3500, or 4000 mW or any range between any of these values or possibly higher or lower.

In various implementations, such as the one shown in FIG. 1, the UVC light projection unit 10 has a handle on the top 26 of the housing 20/projection unit. The handle 32 on the top 26 of the UVC light projection unit 10 (e.g., housing 20) is in a position convenient for holding the unit/housing, for example, such that said plurality of light sources 12, 16 project light in a forward direction (e.g., in the Z direction or parallel to the Z axis shown). In various implementations, the handle 32 is elongate having a length (in the longitudinal, e.g., Z direction) that is longer than wide (in X direction) or thick (in Y direction). In various implementations, the length (in the longitudinal or Z direction) of the handle 32 may be parallel to the forward direction (longitudinal or Z direction) and may be parallel to the length of the top 26, bottom 28, sides 30a, 30b or any combination thereof.

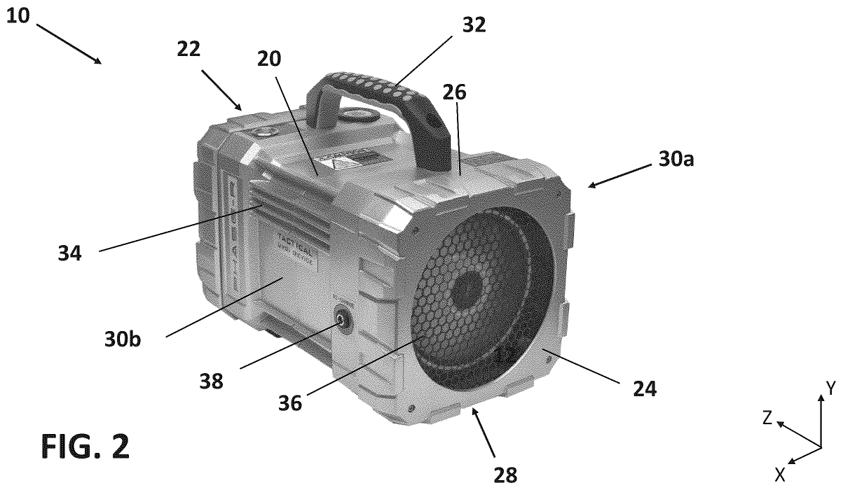

In the example shown in FIG. 1, the housing 20 is generally in the shape of a rectangular prism with the otherwise right angle corners being shaped possibly rounded, for example, to remove the sharp corners. In the design shown in FIG. 1, for example, right angle corners are replaced with bevels 36 or are beveled. The surfaces of the housing 20 also have contours and surface features and are not completely planar. FIG. 1 (as well as FIG. 2), for example, show heat sink fins and/or bladed surfaces 34 on the sides 30a, 30b of the housing. The ridges 24 and grooves therebetween allow for heat to be dissipated. The bladed heatsinks may assist in efficient cooling for the UV LEDs

In various implementation, the length of UVC light projection unit 10 and/or housing 20 is 6, 6.5, 7, 7.5, 8, 8.5, 9, 9.5, 10, 10.5, 11, 11.5, 12, 12.5, 13, 13.5, 14, 14.5 or 15 inches, or any ranges formed by any of these values or possibly larger or smaller. The height maybe 2, 2.5, 3, 3.5, 4, 4.5, 5, 5.5, 6, 6.5, 7, 7.5, or 8 inches, or any ranges formed by any of these values or possibly larger or smaller. The width may be 3, 3.5, 4, 4.5, 5, 5.5, 6, 6.5, 7, 7.5, 8, 8.5, 8, 9.5, 10, 10.5, 11, 11.5, or 12 inches, or any ranges formed by any of these values, or possibly larger or smaller.

As shown in FIG. 2, in various implementations a fan 36 may included to provide for cooling of the UV LEDs. In some designs, this fan 36 may be on the back 24 of the housing 20.

For various designs, the mobile UVC light projection unit 10 comprise components made from different materials. For example, some components are made of metal while other components are made from polymer such as plastic. For example, the housing may be metal or plastic, and, the handle may be plastic. Rubber may also be included, for example, in the handle. This rubber may provide a stronger grip and make it easier for the user not to drop the unit 10. The integration of different materials may allow the UVC light projection unit 10 to be lighter, which may provide for more comfortable use by a user who is carrying the device. For example, while the UV light projection unit 10 comprises a Battery Support Tray, an LED Diode Baseplate (PCB), and Thermal management Heatsink(s) comprising aluminum, other components comprises nylon. Likewise, the UV light projection unit 10 comprises metal and plastic components such as metal (e.g., aluminum) and nylon. As a result, the weight may be 2, 2.5, 3, 3.5, 4, 4.5, 5, 5.5, 6, 6.5 7, 7.5, 8, 9, or 10 lbs. or any range formed by any of these values or may be lighter or heavier, for example, depending on the size and design. Consequently, in some cases, the UVC light projection unit 10 described herein can advantageously be compact, lightweight, convenient to use or any combination of these.

The UVC light projection unit 10 may be rechargeable. For example, the UVC light projection unit 10 may include a battery such as a LiFePO4 or lithium iron phosphate battery that can be recharged. Accordingly, the UVC light projection unit 10 may be powered by a rechargeable LiFePO4 or lithium iron phosphate battery power system. FIG. 2 (as well as FIG. 1) show a recharging port 38 for receiving electrical power such as from a charger.

The UVC light projection unit 10 also comprises an ON/OFF Switch 37 comprising a button made from aluminum that can be pressed to activate the unit. The ON/OFF switch 37 includes with blue LED indicator (which may backlight the button). Other variations, however, are possible.

The UVC light projection unit 10 also comprises Battery Charge Indicator 39. This battery charge indicator includes brass connectors. Other designs, however, may be employed.

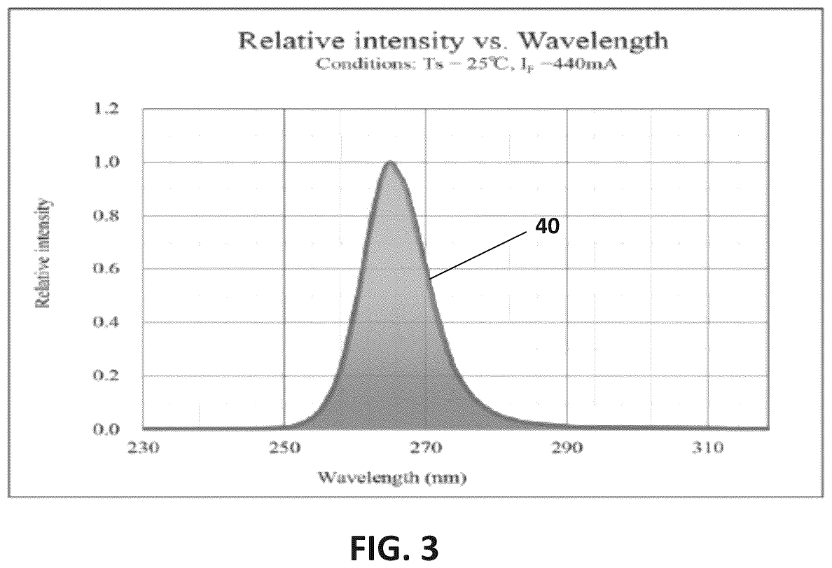

Various designs comprise UVC light sources 12 such as shown in FIGS. 1 and 2 that have a spectral distribution such as shown in FIG. 3. FIG. 3 is a plot on axis of intensity (in relative units) and wavelength (in nanometers) showing the wavelength distribution of light output by the UVC light sources 12 and/or UVC LEDs 14. This intensity versus wavelength curve 40 has a wavelength peak at 265 nm. This distribution 40 also has a full width half maximum (FWHM) from about 260.5 nm to 271 nm. Accordingly, in various implementations, the UVC light source(s) 12 and/or UVC LED(s) 14 may, for example, be configured to emit more than 50%, 60%, 70%, 80%, 90%, 95%, 97%, 98% or 99% of the light output therefrom (or any range formed by any of these percentages) in the wavelength range from 250, 251, 252, 253, 254, 255, 256, 257, 258, 259, 260, 261, 262, 263, 264 or 265 to 266, 267, 268, 269, 270, 271, 272, 273, 274, 276, 278, 279, 280, 283, 285, 287, 290 nm or any range between any of these wavelength values such as 250 nm to 280 nm or 258 to 274 nm or 260 to 280 or 260 to 270 nm or 260 to 271 nm or 263 nm to 267 nm, 265 nm to 275 nm or at 265 nm. In certain designs, percentage values and/or wavelength values are outside these ranges are also possible.

The UVC light sources 12, the visible light sources 16 or both may include optical elements disposed to receive light from the respective UVC LEDs 14, the visible LEDs 18. These optical elements may reduce divergence of light emitted from the LEDs and/or may collimate light emitted from the LEDs.

In some implementations, the optical element comprises a refractive optical element such as a lens that receives light from the LED and transmits light from the LED. In some implementations, the optical element comprises a reflective optical element that receives light from the LED and reflect light from the LED. In some implementations, the optical element comprises both a lens that receives light from the LED and a reflective optical element that receives light from the LED. In some designs, the optical element is configured such that the lens that receives light from the LED and the reflective optical element receives light from the LED transmitted through the lens. Other configurations, however, are possible.

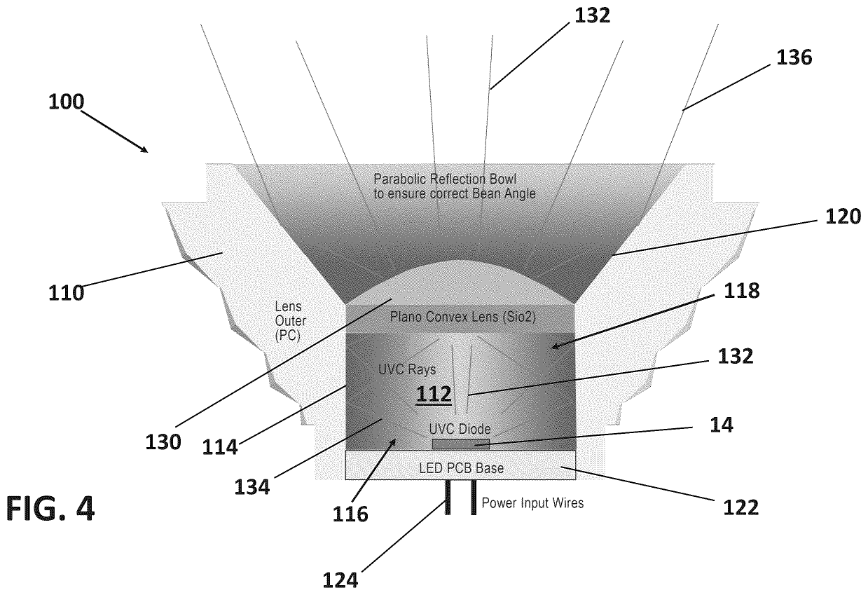

FIGS. 4-7 show an example optical element 100 that can be used for both the UVC light sources 12 and the visible light sources 16. The optical element 100 comprise a body 110 such as shown in FIG. 4. In various implementations, the body 110 comprises plastic such as polycarbonate. In various designs, however, since the optical element 100 will be used in the UVC light source 12, the plastic or polycarbonate material comprises a composition configured to reduce degradation of the plastic or polycarbonate material with exposure to the UVC light. In particular, in various designs, the body 110 comprise plastic or polycarbonate and may comprise heat resistant polycarbonate. One example of polycarbonate material that may be used is formed using an additive comprising tetramethylbisphenol A,

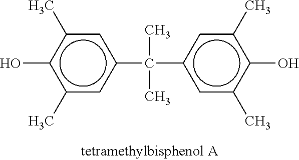

although other additives may be employed. In various implementations, the body material reflects UVC light or at least UVC light having a wavelength(s) that is the same as the wavelength(s) of light output by the UVC LED 14. In various implementations, this same body material may be optically transmissive or transparent to visible light such as visible light having the wavelength(s) of the visible LEDs.

In the example shown in FIG. 4, a channel or neck 112 is within the body 110. The channel 112 has sidewalls 114, which comprise inner surfaces of the body 110. The channel 112 has proximal and distal ends 116, 118. The optical element 100 also includes a concave surface 120 on the inside of the body 112. The concave surface 120 is narrower toward the distal end 118 of the channel 112 and widens with distance from the channel.

A UVC LED 14 is shown at the proximal end 116 of the channel 112.

The UVC LED 14 is mounted on a platform such as a printed circuit board (PCB) 122. Power input leads 124 for providing electrical power to the LED 14 are also shown. Other configurations, however, are possible.

The optical element 100 further comprise a lens 130 disposed to receive UVC light from the UVC LED 14 to transmit UVC light received from said UVC LED. In the example shown, the lens 130 is located at the distal end 118 of the channel 112. In various implementations, the lens 130 has a focal length such as a positive focal length. Additionally, in various implementations, the lens 130 is disposed a distance from the UVC LED 14 with respect to the focal length such that the lens reduces divergence of light received from the UVC LED. Light from the UVC LED may diverge widely (e.g., 120° 140°, 150°, 160°, 170°, 180° or any range between any of these values or possible less). The optical power and position (e.g., distance from UVC LED 14) of the lens 130 may be such that the divergence of the UVC output by the UVC LED is reduced. In some designed, for example, the lens 130 has positive optical power and focal length and is configured to be a focal length from said UVC LED 14. The channel 112 has a length and this length may be configured to position the lens 130 such a distance from the UVC LED 14 to provide a reduction of the divergence angle of UVC light emitted by the UVC LED.

As illustrated, in some designs, the lens 130 comprises a plano-convex lens. The lens 130 may also comprises an aspheric lens having at least one aspheric refractive surface.

The lens 130 is optically transmissive to UVC light, for example, of wavelengths emitted by the UVC light source 12 such as the peak wavelength. Accordingly, in various implementations, the lens 130 is optically transmissive to 220 to 290 nm, 250 nm to 290 nm or 260 nm to 290 nm, 260 to 280 nm or any range formed by any of these values. In some implementations, the lens 130 comprises fused silica.

As discussed above, in various implementations, the lens may comprise fused silica glass having a transmittance (e.g., internal transmittance or transmittance corrected to eliminate the effects of scattering and of reflection from surfaces) of UVC light with 245-280 nm wavelength of least 95% for a 10 mm thickness of the fused silica glass although in other implementations this transmittance is at least 50%, 55%, 60%, 65%, 70%, 75%, 80%, 85%, 90%, 94%, 96%, 98%, 99%, 99.9%, or 100% or any range formed by any of these values or possibly more or less. In various implementations, the OH (e.g., Hydroxyl) content is not larger than 5 ppm although in other implementations the OH content is not larger than 0.05 ppm, 0.1 ppm, 0.50 ppm, 1 ppm, 2 ppm, 3 ppm, 4 ppm, 10 ppm, 20 ppm, 30 ppm, 40 ppm, 50 ppm, 60 ppm, 70 ppm, 80 ppm, 90 ppm, 100 ppm, 125 ppm, 150 ppm, 200 ppm, 300 ppm, 400 ppm, 500 ppm or any range formed by any of these values or possibly more or less. Additionally, in various implementations, a content of Li, Na, K, Mg, Ca and Cu each are smaller than 0.1 ppm although in some implementations the content of any one or more possibly each of Li, Na, K, Mg, Ca and Cu are smaller than 0.001 ppm, 0.005 ppm, 0.01 ppm, 0.05 ppm, 0.2 ppm, 0.3 ppm, 0.4 ppm, 0.5 ppm, 0.6 ppm, 0.7 ppm, 0.8 ppm, 0.9 ppm, 1 ppm, 1.25 ppm, 1.50 ppm, 2.00 ppm, 3.00 ppm, 4.00 ppm, 5.00 ppm or any range formed by any of these values or possibly more or less.

In some case, the glass may be fabricated by crystobalitizing powdery silica raw material and then, fusing the crystobalitized silica material in a non-reducing atmosphere. However, the method of manufacture should not be so limited.

FIG. 4, for example show some rays of UVC light emitted by the UVC LED 14 being transmitted through the lens 130. Some UVC light emitted by the UVC LED 14 (as represented by rays 134) reflects from the sidewalls 114 of the channel 112. As discussed above, in various implementations, the body material reflects UVC light or at least UVC light having a wavelength that same as the wavelength of light output by the UVC LED 14. Accordingly, UVC light 134 from the UVC LED 14 may reflect from the sidewalls 114 of the channel 112 formed by the body 110 comprising this body material that is reflective to the UVC light. Also, as discussed above, the UVC LED 14 emits UVC light having a wide divergence angle and thus some of the UVC light will be incident on and reflect from the sidewalls 114.

FIG. 4 also shows light (represented by rays 136) transmitted through the lens 130 reflecting from the concave surface 120 on the inside of the body 110 of the optical element 100. As discussed above, in various implementations, the body material reflects UVC light or at least UVC light having a wavelength that same as the wavelength of light output by the UVC LED 14. Accordingly, UVC light 136 from the UVC LED 14 may reflect from the concave surface 120 on the inside of the body 110, which comprises this body material that is reflective to the UVC light. This concave surface 120 may have a shape that reduces divergence of the UVC light emitted by the UVC LED 14 and may contribute to the collimation of UVC light from the UVC LED. This concave surface 120 may be curved as shown. This concave surface 120 may be spherical or aspheric in shape. This surface 120 may, in some cases, comprise a parabolic reflector.

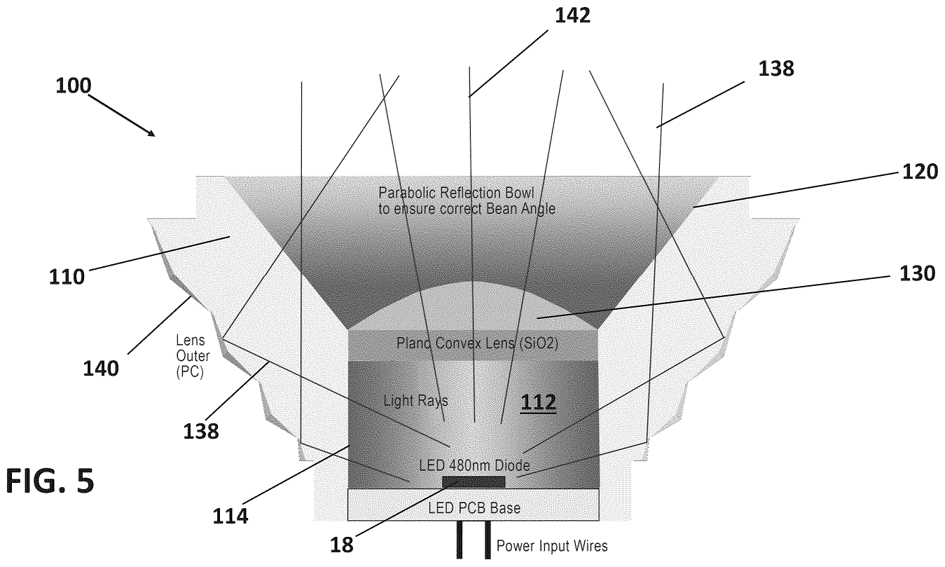

In various implementations, this same optical element 100 as shown in FIG. 4 may alternatively be used in the visible light sources 16 having visible LEDs. FIG. 5 shows the optical element 100 of FIG. 4 with a visible LED 18 instead of a UVC LED 14. As discussed above, although the material comprising the body 110 reflects UVC light or at least UVC light having a wavelength that is the same as the wavelength of light output by the UVC LED 14, this same body material may be optically transmissive or transparent to visible light such as visible light having the wavelength of the visible LEDs 18. Likewise, visible light from the visible LED 18 that is incident on the sidewalls 114 (as represented by ray 138), are transmitted through the sidewall and propagate within the body. The body 110, however, may have an outer surface 140 that is shaped such that visible light incident on and transmitted through the sidewalls 114 (e.g., ray 138) is reflected by total internal reflection by the outer surface/air interface. Likewise, light emitted by the visible LED 18 at a large angle, may be reflected by the outer surface 140 forward. Such a design, may therefore potentially assist in reducing the divergence of light emitted by the visible LED 18. Similarly, in various implementations, the out surface 140, which may also be concave, may contribute to collimation of the visible light from the visible LED 18.

FIG. 5 additionally shows visible light emitted by the visible LED 18 (as represented by ray 142) being transmitted through the lens 130. In various implementations, the lens 130 reduces divergence of the visible light emitted by the visible LED 18. This lens 130 may thus contribute to the collimation of the visible light beam.

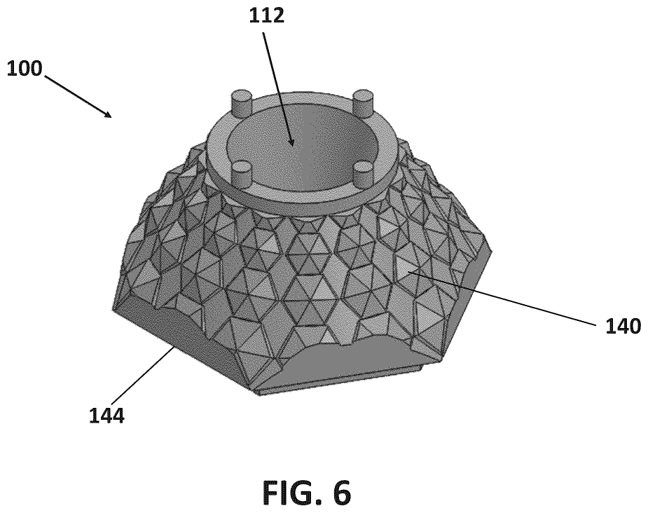

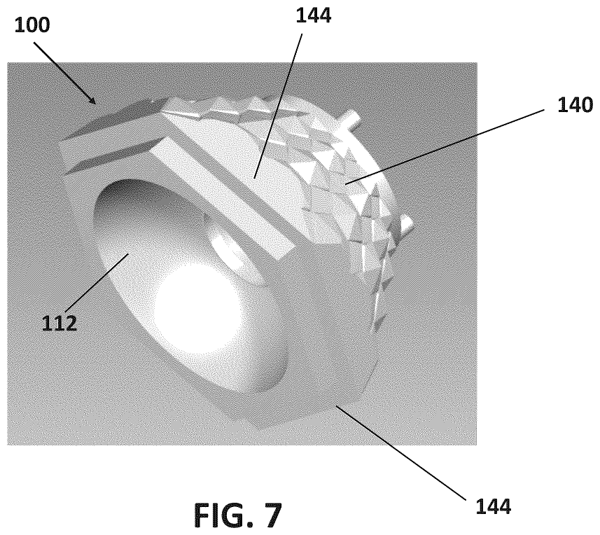

FIGS. 6 and 7 are different perspective views of the optical element 100 shown in FIGS. 4 and 5. FIG. 6 shows outer surface 140 having a pattern thereon. In this example, the pattern is a repeating pattern. In particular, this pattern is a hexagonal pattern. Other patterns may be employed in other designs. Also, the outer surface 140 need not be patterned in some cases. FIG. 6 also shows a design wherein the body 110 has a hexagonal shaped outer perimeter at or proximal to the distal end of the body formed by six flat side portions 144. As discussed above, in various implementations, the array of light sources 12, 16 is also arranged in a hexagonal array.

FIG. 7 is another perspective view of the optical element of FIGS. 4 and 5 showing the concave surface 112 that is reflective to UVC light emitted by the UVC LED 14. The surface 120 is curved and may be spherical or aspheric. In some implementations, this concave surface 120 is parabolically shaped.

As discussed above, the optical element 100 shown in FIG. 5 is the same optical element shown in FIG. 4 but used with a visible LED 18 instead of a UVC LED 14. This optical element 100 is designed to operate with both types of LEDs 14, 18. Having the same design of the optical element 100 for use with both the UVC LEDs 14 and the visible LEDs can simply manufacturing and inventorying as the same component can be used for both the UVC light sources 12 and the visible light sources 16. As the optical elements 100 for both UVC light sources 12 and the visible light sources 16 are interchangeable during manufacture, the UVC LEDs 14 and visible LEDs 16 can be outfitted with the same type of optical element having the same shape and comprising the same material. The number of different types of components used in manufacture and stored in inventory can thereby be reduced.

In various implementations, the UVC light projection unit 10 is configured to emit UVC light of sufficient power to destroy or disable bacteria and/or viruses on a surface to which the UVC light is directed. For example, in various implementations, the UVC light projection unit 10 and the UVC light sources 12 therein emit sufficient radiation to kill or disable most (e.g., at least 20%, 30%, 40%, 50%, 60%, 70%, 80%, 90%, 95%, 98%, 99%, or 100%) of the bacteria and/or viruses on a surface that is 6 inches, 1 foot, 2 feet, or 3 feet from the UVC light projection unit 10 and/or the UVC light sources 12 or LEDs 14 or in any range between any of these percentages or distances and possible farther away. In various implementations, these viruses and/or bacteria may be killed or disabled in within 15 seconds, 12 seconds, 10 seconds, 9 seconds, 8 seconds, 7 seconds, 6 seconds, 5 seconds, 4 seconds, 3 second, 2 second, 1 second or less or in any range between any of these values. In various implementations, the UVC light may illuminate an area of from 0.5 to 4 square feet (e.g., 0.1, 0.2, 0.3, 0.5, 0.8, 1, 1.5, 2, 2.5, 3, 3.5, 4, 4.5, 5, 6, 7, or 8, square feet or any range formed by any of these values or possible larger or smaller) and kill or disable the viruses and bacteria in these short times with this effectiveness. For example, in some implementations, the UVC light projection 10 unit can kill or disable most (e.g., at least 20%, 30%, 40%, 50%, 60%, 70%, 80%, 90%, 95%, 98%, 99%, or 100% or any range formed by any of these values) of the bacteria or viruses on, and thus potentially sanitize, a surface having an area of 4 square feet that is 3 feet away from the UVC LEDs 14 and/or the UVC light projection unit 10 within 10 seconds possibly within 2, 3, 4, 5 or 6 seconds or any range between any of these values.

Accordingly, the UVC light projection unit 10 described herein may be effective at providing for partial or full sterilization and/or inactivation of surfaces. This device 10 thus stands to be an effective compact mobile tool for combating infectious viruses and disease carrying bacteria such as in hospitals, airplanes, and other public areas. This unit 10 may also be used in other scenarios, such as for biohazard cleanup, for sanitizing military equipment contaminated due to germ warfare, etc., by destroying or at least degrading the potency of certain biological contaminants.

### Examples

The following is a numbered list of example embodiments that are within the scope of this disclosure. The example embodiments that are listed should in no way be interpreted as limiting the scope of the embodiments. Various features of the example embodiments that are listed can be removed, added, or combined to form additional embodiments, which are part of this disclosure.

### Part I

1. A UVC light projection unit for providing UVC illumination, said UVC light projection unit comprising:
   a housing having a front and back, top and bottom and sides;
   a plurality of light sources supported by said housing, said plurality of light sources comprising a first group of light sources comprising a plurality of UVC light emitting diodes (LED) and a second group of light sources comprising a plurality of visible light emitting diodes configured to project UVC light and visible light forward;
   a handle closer to the top of said housing than the bottom or sides, said handle for holding said housing such that said plurality of light sources project light in a forward direction; and
   a fan disposed to provide cooling for said plurality of light sources.
2. The UVC light projection unit of Example 1, wherein said plurality of light sources are directed in a forward longitudinal direction and said handle has a length in said longitudinal direction, said handle being longer than thick and longer than wide.
3. The UVC light projection unit of Example 1, wherein both said housing and said handle are elongated along a longitudinal direction.
4. The UVC light projection unit of Example 1, wherein said housing is longer in a longitudinal direction than wide in a transverse direction and longer in said longitudinal direction than high in a direction orthogonal to said longitudinal and transverse directions.
5. The UVC light projection unit of Example 4, wherein said handle is longer in said longitudinal direction than wide in said transverse direction and longer in said longitudinal direction than thick in said direction orthogonal to said longitudinal and transverse directions.
6. The UVC light projection unit of any of Example 2-5, wherein said plurality of light sources point in said longitudinal direction.
7. The UVC light projection unit of any of Examples 2-6, wherein said plurality of light sources project light in a forward direction parallel to said longitudinal direction.
8. The UVC light projection unit of any of Example 2-7, wherein said plurality of light sources are configured to direct most of said UVC light within ±45° of said longitudinal direction.
9. The UV The UVC light projection unit of any of Example 2-8, wherein said plurality of light sources are configured to direct light in a beam centered within ±5° of said longitudinal direction.
10. The UV The UVC light projection unit of any of Example 2-9, wherein said plurality of light sources are configured to direct light in a beam centered around said longitudinal direction.
11. The UVC light projection unit of any of the examples above, wherein said top, bottom and sides have a length in a longitudinal direction and said top and bottom have a width in a transverse direction, and wherein said top and bottom of said housing is longer than wide.
12. The UVC light projection unit of any of the examples above, wherein said sides have a height orthogonal to said longitudinal and transverse directions and said sides are longer than high.
13. The UVC light projection unit of Example 12, wherein said handle is elongated in said longitudinal direction so as to have a length in said longitudinal direction that is longer than the width of said handle in said transverse direction or the thickness of said handle in said direction orthogonal to said longitudinal and transverse directions.
14. The UVC light projection unit of any of the examples above, wherein said housing has a shape of a contoured rectangular prism.
15. The UVC light projection unit of any of the examples above, wherein said housing comprises metal.
16. The UVC light projection unit of any of the examples above, wherein said housing comprises plastic.
17. The UVC light projection unit of any of the examples above, further comprising bladed surfaces or fins on said housing.
18. The UVC light projection unit of any of the examples above, wherein said fan is in the back of said housing.
19. The UVC light projection unit of any of the examples above, wherein said handles comprise plastic.
20. The UVC light projection unit of any of the examples above, wherein said handles includes rubber texturing that enhances grip.
21. The UVC light projection unit of any of the examples above, wherein said front and back have rectangular or square profiles.
22. The UVC light projection unit of any of the examples above, wherein said front and back have rounded or beveled corners
23. The UVC light projection unit of any of the examples above, wherein said top and bottom have rectangular profiles.
24. The UVC light projection unit of any of the examples above, wherein said top and bottom have rounded or beveled corners
25. The UVC light projection unit of any of the examples above, wherein said sides have rectangular profiles.
26. The UVC light projection unit of any of the examples above, wherein said sides have rounded or beveled corners.
27. The UVC light projection unit of any of the examples above, wherein said plurality of light sources are arranged in an array.
28. The UVC light projection unit of any of the examples above, wherein said plurality of light sources are arranged in a hexagonal array.
29. The UVC light projection unit of any of the examples above, wherein said plurality of UVC LEDs are configured with respective lenses configured to receive UVC light from said UVC LEDs and project said UVC light forward.
30. The UVC light projection unit of any of the examples above, wherein said plurality of UVC LEDs are configured with respective reflectors configured to receive UVC light from said UVC LEDs and project said UVC light forward.
31. The UVC light projection unit of any of the examples above, wherein said plurality of UVC LEDs are configured with respective optical elements comprising a combination of a lens and a reflector configured to receive UVC light from said UVC LED and project said UVC light forward.
32. The UVC light projection unit of any of the examples above, wherein said plurality of visible LEDs are configured with respective lenses configured to receive visible light from said visible LEDs and project said visible light forward.
33. The UVC light projection unit of any of the examples above, wherein said plurality of visible LEDs are configured with respective reflectors configured to receive visible light from said visible LEDs and project said visible light forward.
34. The UVC light projection unit of any of the examples above, wherein said plurality of visible LEDs are configured with respective optical elements comprising a combination of a lens and a reflector configured to receive visible light from said visible LED and project said visible light forward.
35. The UVC light projection unit of any of the examples above, further comprising one or more rechargeable batteries.
36. The UVC light projection unit of any of the examples above, wherein said UVC light projection unit is configured to be re-charged, said housing including a jack for providing electrical power for recharging.
37. The UVC light projection unit of any of the examples above, wherein said plurality of UVC light sources output light having a radiant flux in the range of from 10 to 1000 mW.
38. The UVC light projection unit of any of the examples above, wherein said plurality of UVC light sources output light having a radiant flux in the range of from 100 to 300 mW.
39. The UVC light projection unit of any of the examples above, wherein said plurality of UVC LEDs output light having a radiant flux in the range of from 10 to 1000 mW.
40. The UVC light projection unit of any of the examples above, wherein said plurality of UVC LEDs output light having a radiant flux in the range of from 100 to 600 mW.
41. The UVC light projection unit of any of the examples above, wherein said plurality of UVC LEDs are configured to emit light having a peak wavelength in the range of 260 to 280 nm.
42. The UVC light projection unit of any of the examples above, wherein said plurality of UVC light sources are configured to emit light having a peak wavelength in the range of 260 to 280 nm.
43. The UVC light projection unit of any of the examples above, wherein at least one of said UVC light sources further comprises a lens configured to receive UVC light from said UVC LED and to transmit UVC light received from said UVC LED.
44. The UVC light projection unit of Example 43, wherein said lens has a focal length and is configured to be a focal length from said UVC LED.
45. The UVC light projection unit of Example 43, wherein said lens has a focal length and is disposed a distance from said UVC LED with respect to said focal length such that said lens reduces divergence of light received from said UVC LED.
46. The UVC light projection unit of any of Examples 43-45, wherein said lens comprises an aspheric lens comprises a positive lens.
47. The UVC light projection unit of any of Examples 43-46, wherein said lens comprises an aspheric lens having an aspheric refractive surface.
48. The UVC light projection unit of any of Examples 43-47, wherein said lens comprises fused silica.
49. The UVC light projection unit of any of Examples 43-48, wherein said lens comprises a plano-convex lens.
50. The UVC light projection unit of any of Examples 1-42, wherein at least one of said UVC light sources further comprises an optical element disposed to receive UVC light from said respective UVC LED, said optical element comprising a body comprising material reflective to said UVC light and a UVC reflective surface on said body configured to reflect said UVC light.
51. The UVC light projection unit of Example 50, wherein said UVC reflective surface comprises a curved reflective surface.
52. The UVC light projection unit of Example 50 or 51, wherein said UVC reflective surface comprises a concave reflective surface.
53. The UVC light projection unit of any of Examples 50-52, wherein said UVC reflective surface comprises comprise reflective surface that extends arcuately increasing in width with longitudinal distance from said UVC LED.
54. The UVC light projection unit of any of Examples 50-53, wherein said UVC reflective surface has a spherical shape.
55. The UVC light projection unit of any of Examples 50-53, wherein said UVC reflective surface has an aspherical shape.
56. The UVC light projection unit of any of Examples 50-53, wherein said UVC reflective surface comprises a parabolic reflector.
57. The UVC light projection unit of any of Examples 50-56, wherein said body has a hexagonal shaped outer perimeter at said distal end of said body formed by six flat side portions.
58. The UVC light projection unit of any of Examples 50-57, wherein said body has an exterior surface having a pattern thereon.
59. The UVC light projection unit of any of Examples 50-58, wherein said body has an exterior surface having a repeating pattern thereon.
60. The UVC light projection unit of any of the Examples 50-59, wherein said body has an exterior surface having a hexagonal pattern thereon.
61. The UVC light projection unit of any of Examples 50-60, wherein said material comprises a composition configured to reduce degradation of said material with exposure to said UVC light.
62. The UVC light projection unit of any of Examples 50-61, wherein said material comprises a polycarbonate.
63. The UVC light projection unit of any of Examples 50-62, wherein said material comprises heat resistant polycarbonate
64. The UVC light projection unit of any of Examples 50-63, wherein said tetramethylbisphenol A comprises an additive for said material.
65. The UVC light projection unit of any of Examples 50-64, further comprising a lens configured to receive UVC light from said UVC LED and to transmit UVC light received from said UVC LED.
66. The UVC light projection unit of Example 65, wherein said lens has a focal length and is configured to be a focal length from said UVC LED.
67. The UVC light projection unit of Example 65, wherein said lens has a focal length and is disposed a distance from said UVC LED with respect to said focal length such that said lens reduces divergence of UVC light received from said UVC LED.
68. The UVC light projection unit of any of the examples above, wherein at least one of said visible light sources further comprises a lens configured to receive visible light from said visible LED and to transmit visible light received from said visible LED.
69. The UVC light projection unit of Example 68, wherein said lens has a focal length and is configured to be a focal length from said visible LED.
70. The UVC light projection unit of Example 68, wherein said lens has a focal length and is disposed a distance from said visible LED with respect to said focal length such that said lens reduces divergence of visible light received from said visible LED.
71. The UVC light projection unit of any of Examples 68-70, wherein said lens comprises an aspheric lens comprises a positive lens.
72. The UVC light projection unit of any of Examples 68-71, wherein said lens comprises an aspheric lens having an aspheric refractive surface.
73. The UVC light projection unit of any of Examples 68-72, wherein said lens comprises fused silica.
74. The UVC light projection unit of any of Examples 68-73, wherein said lens comprises a plano-convex lens.
75. The UVC light projection unit of any of Examples 1-67, wherein at least one of said visible light sources further comprises an optical element disposed to receive visible light from said respective visible LED, said optical element comprising a body comprising material transmissive to said visible light and an exterior surface on said body configured to reflect said visible light by total internal reflection.
76. The UVC light projection unit of Example 75, wherein said exterior surface comprises a concave surface.
77. The UVC light projection unit of any of Examples 75, wherein said exterior surface extends arcuately increasing in width with longitudinal distance from said visible LED.
78. The UVC light projection unit of any of Examples 75-77, wherein said body has a hexagonal shaped outer perimeter at said distal end of said body formed by six flat side portions.
79. The UVC light projection unit of any of Examples 75-78, wherein said exterior surface has a pattern thereon.
80. The UVC light projection unit of any of Examples 75-79, wherein said exterior surface has a repeating pattern thereon.
81. The UVC light projection unit of any of the Examples 75-80, wherein said exterior surface having a hexagonal pattern thereon.
82. The UVC light projection unit of any of Examples 75-81, wherein said material comprises a composition configured to reduce degradation of said material with exposure to said UVC light.
83. The UVC light projection unit of any of Examples 75-82, wherein said material comprises a polycarbonate.
84. The UVC light projection unit of any of Examples 75-83, wherein said material comprises heat resistant polycarbonate.
85. The UVC light projection unit of any of Examples 75-84, wherein tetramethylbisphenol A is an additive in said material.
86. The UVC light projection unit of any of Examples 75-85, further comprising a lens configured to receive visible light from said visible LED and to transmit visible light received from said visible LED.
87. The UVC light projection unit of Example 86, wherein said lens has a focal length and is configured to be a focal length from said visible LED.
88. The UVC light projection unit of Example 86, wherein said lens has a focal length and is disposed a distance from said visible LED with respect to said focal length such that said lens reduces divergence of visible light received from said visible LED.
89. The UVC light projection unit of any of Examples 75-88, wherein said optical element in said UVC light source has the same design as the optical element in the visible light source.
90. The UVC light projection unit of any of Examples 68-74 or 86-88, wherein said lens in said UVC light source has the same design as the lens in the visible light source.
91. The UVC light projection unit of any of Examples 75-88, wherein said body of the optical element in said UVC light source has the same shape and comprises the same material design as said body of the optical element in the visible light source.
92. The UVC light projection unit of any of Examples 68-74 or 86-88, wherein said lens in said UVC light source has the same shape and comprise the same material as the lens in the visible light source.
93. The UVC light projection unit of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
94. The UVC light projection unit of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
95. The UVC light projection unit of any of the above, wherein said lens comprises fused silica wherein the OH content is not larger than 5 ppm.
96. The UVC light projection unit of any of the above, wherein said lens comprises fused silica wherein the content of each of Li, Na, K, Mg, Ca and Cu are smaller than 0.1 ppm.
97. The UVC light projection unit of any of the above, wherein said lens comprises fused silica fabricated by crystobalitizing powdery silica raw material and fusing the crystobalitized silica material in a non-reducing atmosphere.
98. The UVC light projection unit of any of the examples above, wherein said plurality of UVC LEDs output light having a radiant flux in the range of from 200 to 400 mW.
99. The UVC light projection unit of any of the examples above, wherein said UVC light projection unit emits sufficient radiation to kill or disable most of the bacteria and/or viruses on a surface that is 6 inches from the UVC light projection unit within 15 seconds.
100. The UVC light projection unit of any of the examples above, wherein said UVC light projection unit emits sufficient radiation to kill or disable most of the bacteria and/or viruses on a surface that is 1 foot from the UVC light projection unit within 15 seconds.
101. The UVC light projection unit of any of the examples above, wherein said UVC light projection unit comprises a lithium phosphate rechargeable power system powered by a lithium phosphate rechargeable battery.

### Part IIA

1. An UVC light source comprising:
   an UVC light emitting diode (LED) configured to emit UVC light having a wavelength in the range between 250 nm and 280 nm; and
   an optical element configured to receive UVC light from said UV LED, said optical element comprising:
      a body comprising material reflective to said UVC light;
      a channel in said body, said channel having proximal and distal ends, said UVC LED at said proximal end of said channel such that UVC light from said UVC LED propagates to said distal end of said channel;
      a fused silica lens disposed at said distal end of said channel to receive UVC light from said UVC LED coupled into said channel, said fused silica lens having positive optical power; and
      a concave reflective surface on said body, said concave reflective surface disposed about said distal end of said channel, said concave reflective surface configured to reflect UVC light from said UVC LED forward said optical element.
2. The UVC light source of Example 1, wherein said concave reflective surface comprise a curved concave reflective surface.
3. The UVC light source of Example 1 or 2, wherein said concave reflective surface extends arcuately from the distal end of the channel increasing in width with longitudinal distance forward said channel.
4. The UVC light source of any of Examples 1-3, wherein said concave reflective surface has a spherical shape.
5. The UVC light source of any of Examples 1-3, wherein said concave curved reflective surface has an aspherical shape.
6. The UVC light source of any of Examples 1-3, wherein said concave curved reflective surface comprises a parabolic reflector.
7. The UVC light source of any of the examples above, wherein said body has a hexagonal shaped outer perimeter at said distal end of said body formed by six flat side portions.
8. The UVC light source of any of the examples above, wherein said body has an exterior surface having a pattern thereon.
9. The UVC light source of any of the examples above, wherein said body has an exterior surface having a repeating pattern thereon.
10. The UVC light source of any of the examples above, wherein said body has an exterior surface having a hexagonal pattern thereon.
11. The UVC light source of any of the examples above, wherein said lens has a focal length and the channel has a length configured such that said lens is a focal length from said UVC LED.
12. The UVC light source of any of the examples above, wherein said lens has a focal length and the channel has a length with respect to said focal length such that said lens reduces divergence of light received from said UVC LED.
13. The UVC light source of any of the examples above, wherein said material comprises a composition configured to reduce degradation of said material with exposure to said UVC light.
14. The UVC light source of any of the examples above, wherein said material comprises a polymer material.
15. The UVC light source of any of the examples above, wherein said material comprises a polycarbonate.
16. The UVC light source of any of the examples above, wherein said material comprises heat resistant polycarbonate.
17. The UVC light source of any of the examples above, wherein tetramethylbisphenol A is an additive for said material.
18. The UVC light source of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
19. The UVC light source of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
20. The UVC light source of any of the above, wherein said lens comprises fused silica wherein the OH content is not larger than 5 ppm.
21. The UVC light source of any of the above, wherein said lens comprises fused silica wherein the content of each of Li, Na, K, Mg, Ca and Cu are smaller than 0.1 ppm.
22. The UVC light source of any of the above, wherein said lens comprises fused silica fabricated by crystobalitizing powdery silica raw material and fusing the crystobalitized silica material in a non-reducing atmosphere.

### Part IIB

1. A UVC light source comprising:
   an UVC light emitting diode (LED) configured to emit UVC light having a wavelength in the range between 250 nm and 280 nm;
   an optical element disposed to receive UVC light from said UVC LED, said optical element comprising:
      reflective optical element comprising a body comprising material reflective to said UVC light and a reflective surface on said body configured to reflect said UVC light, said material configured to reduce degradation of said material with exposure to said UVC light; and
      a fused silica optically transmissive lens configured to transmit UVC light from said UV LED.
2. The UVC light source of Example 1, wherein said reflective surface comprises a curved reflective surface.
3. The UVC light source of Example 1 or 2, wherein said reflective surface comprises a curved reflective surface.
4. The UVC light source of any of Examples 1-3, wherein said reflective surface extends arcuately increasing in width with longitudinal distance from said UVC LED.
5. The UVC light source of any of Examples 1-4, wherein said reflective surface has a spherical shape.
6. The UVC light source of any of Examples 1-4, wherein said reflective surface has an aspherical shape.
7. The UVC light source of any of Examples 1-4, wherein said reflective surface comprises a parabolic reflector.
8. The UVC light source of any of the examples above, wherein said body has a hexagonal shaped outer perimeter at said distal end of said body formed by six flat side portions.
9. The UVC light source of any of the examples above, wherein said body has an exterior surface having a pattern thereon.
10. The UVC light source of any of the examples above, wherein said body has an exterior surface having a repeating pattern thereon.
11. The UVC light source of any of the examples above, wherein said body has an exterior surface having a hexagonal pattern thereon.
12. The UVC light source of any of the examples above, wherein said material comprises a composition configured to reduce degradation of said material with exposure to said UVC light.
13. The UVC light source of any of the examples above, wherein said material comprises a polymer material.
14. The UVC light source of any of the examples above, wherein said material comprises a polycarbonate.
15. The UVC light source of any of the examples above, wherein said material comprises heat resistant polycarbonate.
16. The UVC light source of any of the examples above, wherein tetramethylbisphenol A is an additive for said material.
17. The UVC light source of any of the examples above, wherein said fused silica lens has a focal length and is configured to be a focal length from said UVC LED.
18. The UVC light source of any of the examples above, wherein said lens has a focal length and is disposed a distance from said UVC LED with respect to said focal length such that said lens reduces divergence of light received from said UVC LED.
19. The UVC light source of any of the examples above, wherein said reflective optical element is configured to receive UVC light transmitted through said fused silica transmissive optical element.
20. The UVC light source of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
21. The UVC light source of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
22. The UVC light source of any of the above, wherein said lens comprises fused silica wherein the OH content is not larger than 5 ppm.
23. The UVC light source unit of any of the above, wherein said lens comprises fused silica wherein the content of each of Li, Na, K, Mg, Ca and Cu are smaller than 0.1 ppm.
24. The UVC light source of any of the above, wherein said lens comprises fused silica fabricated by crystobalitizing powdery silica raw material and fusing the crystobalitized silica material in a non-reducing atmosphere.

### Part IIC

1. A UVC light source comprising:
   an ultraviolet (UV) light emitting diode (LED) configured to emit UVC light having a wavelength in the range between 250 nm and 280 nm;
   optics disposed to receive UVC light from said UVC LED, said optics comprising:
   a reflective optical element comprising a body comprising material reflective to said UVC light and a reflective surface on said body configured to reflect said UVC light.
2. The UVC light source of Example 1, wherein said reflective surface comprises a curved reflective surface.
3. The UVC light source of Example 1 or 2, wherein said reflective surface comprises a concave reflective surface.
4. The UVC light source of any of Examples 1-3, wherein said reflective surface comprises comprise curved reflective surface that extends arcuately increasing in width with longitudinal distance from said UVC LED.
5. The UVC light source of any of Examples 1-4, wherein said reflective surface has a spherical shape.
6. The UVC light source of any of Examples 1-4, wherein said reflective surface has an aspherical shape.
7. The UVC light source of any of Examples 1-4, wherein said reflective surface comprises a parabolic reflector.
8. The UVC light source of any of the examples above, wherein said body has a hexagonal shaped outer perimeter at said distal end of said body formed by six flat side portions.
9. The UVC light source of any of the examples above, wherein said body has an exterior surface having a pattern thereon.
10. The UVC light source of any of the examples above, wherein said body has an exterior surface having a repeating pattern thereon.
11. The UVC light source of any of the examples above, wherein said body has an exterior surface having a hexagonal pattern thereon.
12. The UVC light source of any of the examples above, further comprising a lens configured to receive UVC light from said UVC LED and to transmit UVC light received from said UVC LED.
13. The UVC light source of Example 12, wherein said lens has a focal length and is configured to be a focal length from said UVC LED.
14. The UVC light source of Examples 12, wherein said lens has a focal length and is disposed a distance from said UVC LED with respect to said focal length such that said lens reduces divergence of light received from said UVC LED.
15. The UVC light source of any of the examples above, wherein said material comprises a composition configured to reduce degradation of said material with exposure to said UVC light.
16. The UVC light source of any of the examples above, wherein said material comprises a polymer material.
17. The UVC light source of any of the examples above, wherein said material comprises a polycarbonate.
18. The UVC light source of any of the examples above, wherein said material comprises heat resistant polycarbonate.
19. The UVC light source of any of the examples above, wherein tetramethylbisphenol A is an additive for said material.
20. The UVC light source of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
21. The UVC light source of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
22. The UVC light source of any of the above, wherein said lens comprises fused silica wherein the OH content is not larger than 5 ppm.
23. The UVC light source of any of the above, wherein said lens comprises fused silica wherein the content of each of Li, Na, K, Mg, Ca and Cu are smaller than 0.1 ppm.
24. The UVC light source of any of the above, wherein said lens comprises fused silica fabricated by crystobalitizing powdery silica raw material and fusing the crystobalitized silica material in a non-reducing atmosphere.

### Part IID

1. An optical element configured to receive UVC light from a UVC light emitting diode (LED) or visible light from a visible LED, said optical element comprising:
   a body comprising material reflective to said UVC light;
   a channel in said body, said channel having proximal and distal ends, said UVC LED at said proximal end of said channel such that UVC light from said UVC LED propagates to said distal end of said channel;
      a fused silica lens disposed at said distal end of said channel to receive UVC light from said UVC LED or said visible light from said visible LED coupled into said channel, said fused silica lens having positive optical power; and
      a concave reflective surface on said body, said concave reflective surface disposed about said distal end of said channel.
2. The optical element of Example 1, wherein said concave reflective surface comprises a curved reflective surface.
3. The optical element of any of Examples 1 or 2, wherein said concave reflective surface comprises comprise curved reflective surface that extends arcuately increasing in width with longitudinal distance from said UVC LED or visible LED.
4. The optical element of any of Examples 1-3, wherein said reflective surface has a spherical shape.
5. The optical element of any of Examples 1-3, wherein said reflective surface has an aspherical shape.
6. The optical element of any of Examples 1-3, wherein said reflective surface comprises a parabolic reflector.
7. The optical element of any of the examples above, wherein said body has a hexagonal shaped outer perimeter at said distal end of said body formed by six flat side portions.
8. The optical element of any of the examples above, wherein said body has an exterior surface having a pattern thereon.
9. The optical element of any of the examples above, wherein said body has an exterior surface having a repeating pattern thereon.
10. The optical element of any of the examples above, wherein said body has an exterior surface having a hexagonal pattern thereon.
11. The optical element of any of Examples 1-10, wherein said fused silica lens has a focal length and is configured to be a focal length from said UVC LED or said visible LED.
12. The optical element of any of Examples 1-10, wherein said lens has a focal length and is disposed a distance from said UVC LED with respect to said focal length such that said lens reduces divergence of light received from said UVC LED.
13. The optical element of any of Examples 1-10, wherein said lens has a focal length and is disposed a distance from said visible LED with respect to said focal length such that said lens reduces divergence of light received from said visible LED.
14. The optical element of any of the examples above, wherein said concave reflective surface is configured to reflect UVC light from the UV LED forward said optical element.
15. The optical element of any of the examples above, wherein said body is optically transmissive to said visible light.
16. The optical element of any of the examples above, wherein said body comprises an outer surface angled with respect to said visible LED such that a portion of said visible light emitted from said visible LED at an angle propagates through said body and is reflected from said outer surface and is redirected forward said optical element.
17. The optical element of any of the examples above, wherein said polymer material comprises a composition configured to reduce degradation of said material with exposure to said UVC light.
18. The optical element of any of the examples above, wherein said material comprises a polymer material.
19. The optical element of any of the examples above, wherein said material comprises a polycarbonate.
20. The optical element of any of the examples above, wherein said material comprises heat resistant polycarbonate.
21. The optical element of any of the examples above, wherein tetramethylbisphenol A is an additive for said material.
22. The optical element of any of the above, wherein said lens comprises fused silica having an internal transmittance corrected to eliminate the effects of scattering, absorption and surface reflection of UVC light with 245-280 nm wavelength of at least 90% for fused silica glass having a thickness of 10 mm.
23. The optical element of any of the above, wherein said lens comprises fused silica having an internal transmittance corrected to eliminate the effects of scattering, absorption and surface reflection of UVC light with 245-280 nm wavelength of at least 95% for fused silica glass having a thickness of 10 mm
24. The optical element of any of the above, wherein said lens comprises fused silica wherein the OH content is not larger than 5 ppm.
25. The optical element of any of the above, wherein said lens comprises fused silica wherein the content of each of Li, Na, K, Mg, Ca and Cu are smaller than 0.1 ppm.
26. The optical element of any of the above, wherein said lens comprises fused silica fabricated by crystobalitizing powdery silica raw material and fusing the crystobalitized silica material in a non-reducing atmosphere.

A wide range of variations are possible. Structures, components, and/or feature, for example, can be added, removed, and/or rearranged.

### CONCLUSION

Various embodiments of the present invention have been described herein. Although this invention has been described with reference to these specific embodiments, the descriptions are intended to be illustrative of the invention and are not intended to be limiting. Various modifications and applications may occur to those skilled in the art without departing from the true spirit and scope of the invention.

1. A UVC light projection unit for providing UVC illumination, said UVC light projection unit comprising:
a housing having a front and back, top and bottom and sides;
a plurality of light sources supported by said housing, said plurality of light sources comprising a first group of light sources comprising a plurality of UVC light emitting diodes (LED) and a second group of light sources comprising a plurality of visible light emitting diodes configured to project UVC light and visible light forward;
a handle closer to the top of said housing than the bottom or sides, said handle for holding said housing such that said plurality of light sources project light in a forward direction; and
a fan disposed to provide cooling for said plurality of light sources.

2. (canceled)

3. The UVC light projection unit of claim 1, wherein both said housing and said handle are elongated along a longitudinal direction.

4. (canceled)

5. (canceled)

6. (canceled)

7. The UVC light projection unit of claim 3, wherein said plurality of light sources project light in a forward direction parallel to said longitudinal direction.

8. The UVC light projection unit of claim 3, wherein said plurality of light sources are configured to direct most of said UVC light within ±45° of said longitudinal direction.

9. The UV The UVC light projection unit of claim 3, wherein said plurality of light sources are configured to direct light in a beam centered within ±5° of said longitudinal direction.

10. (canceled)

11. (canceled)

12. (canceled)

13. (canceled)

14. The UVC light projection unit of claim 1, wherein said housing has a shape of a contoured rectangular prism.

15. (canceled)

16. (canceled)

17. The UVC light projection unit of claim 1, further comprising bladed surfaces or fins on said housing.

18. The UVC light projection unit of claim 1, wherein said fan is in the back of said housing.

19. The UVC light projection unit of claim 1, wherein said handles comprise plastic.

20. The UVC light projection unit of claim 1, wherein said handles includes rubber texturing that enhances grip.

21. The UVC light projection unit of claim 1, wherein said front and back have rectangular or square profiles.

22. The UVC light projection unit of claim 1, wherein said front and back have rounded or beveled corners

23. (canceled)

24. (canceled)

25. The UVC light projection unit of claim 1, wherein said sides have rectangular profiles.

26. The UVC light projection unit of any of claim 1, wherein said sides have rounded or beveled corners.

27. (canceled)

28. The UVC light projection unit of claim 1, wherein said plurality of light sources are arranged in a hexagonal array.

29. The UVC light projection unit of claim 1, wherein said plurality of UVC LEDs are configured with respective lenses configured to receive UVC light from said UVC LEDs and project said UVC light forward.

30. (canceled)

31. The UVC light projection unit of claim 1, wherein said plurality of UVC LEDs are configured with respective optical elements comprising a combination of a lens and a reflector configured to receive UVC light from said UVC LED and project said UVC light forward.

32. (canceled)

33. (canceled)

34. (canceled)

35. (canceled)

36. (canceled)

37. (canceled)

38. (canceled)

39. (canceled)

40. (canceled)

41. (canceled)

42. (canceled)
